(19) United States
(12) Patent Application Publication
(10) Pub. No.: US 2015/0086622 A1
(43) Pub. Date: Mar. 26, 2015

(54) GROWTH ENHANCEMENT OF INFANTS

(71) Applicant: NUTRINIA LTD., Nazareth Illit (IL)

(72) Inventors: Naim Shehadeh, Kfar-Yasif (IL); Aviv Hanien, Haifa (IL); Chen Porat, Kiryat Tiv'on (IL); Raanan Shamir, Herzlia (IL); Lora Eshkar-Sebban, Beit Hashmonai (IL); Michal Devir, Kfar Vitkin (IL); Michal Olshansky, Tel Aviv (IL)

(21) Appl. No.: 14/394,785

(22) PCT Filed: Apr. 17, 2013
(86) PCT No.: PCT/IL2013/050333

§ 371 Date: Oct. 16, 2014

Related U.S. Application Data
(60) Provisional application No. 61/625,697, filed on Apr. 18, 2012.

(51) Int. Cl. A23L 1/29; A61K 9/48; A61K 38/28

(52) U.S. Cl. CPC A23L 1/296; A61K 38/28; A61K 9/48
USPC 424/451; 514/5.3; 426/2

(57) ABSTRACT

The present invention relates to compositions and methods for enhancing the growth of infants. Particularly, the present invention discloses the use of insulin for promoting the growth of low birth weight infants, including preterm infants and small for gestational age (SGA) infants over the expected rate.

### FIELD OF THE INVENTION

The present invention relates to compositions and methods for enhancing the growth of infants, particularly to insulin-supplement and formulae comprising same for preterm infants and small for gestational age (SGA) infants, which is effective in promoting the newborn growth.

### BACKGROUND OF THE INVENTION

Breastfeeding is acknowledged as the natural and advisable way of supporting the healthy growth and development of infants due to its nutritional and immunological advantages (ESPGHAN Committee on Nutrition: Agostoni C. et al. Breast-feeding: A commentary by the ESPGHAN Committee on Nutrition. J Pediatr Gastroenterol Nutr 2009. 49:112-25). Breast milk provides the most suitable diet for infant's nutritional requirements. It also provides the infant with immune protection against a wide range of infection related diseases (Shulman R. J. Pediatr Res 1990. 28:171-5), and is also found to provide long-term benefits in the area of certain cognitive developments. It is also well known that the composition of human milk changes over the first few weeks following delivery of an infant. Human milk is referred to as colostrum during the first 5 days after birth, transition milk during days 6-14 after birth, and mature milk thereafter. During each stage of lactation, the corresponding human milk composition differs considerably. Colostrum and transition milk, for example, have lower caloric densities than mature milk, as well as higher protein and lower carbohydrate concentrations. Vitamin and minerals as well as hormone concentrations also vary in the three defined human milk groups. However, breastfeeding is not always possible, particularly when babies are born preterm or with a low birth weight.

There are many different infant nutritional formulas that are commercially available or otherwise known in the infant formula art. These infant formulae comprise a range of nutrients to meet the nutritional needs of the growing infant, and typically include lipids, carbohydrates, protein, vitamins, minerals, and other nutrients helpful for optimal infant growth and development. While an effort is made to make the commercial infant formulae similar in composition to mature human milk, they are not identical, typically due to the formula processing conditions. One of the components missing from commercial infant formulae is insulin, known to be present in its active form in maternal milk.

Observations on lactating dams and suckling rats have shown that mammalian milk insulin is biologically active, and that immature enterocytes have an increased responsiveness to the insulin (Buts J. P. et al., J Pediatr Gastroenterol Nutr 1997. 25:230-2). Insulin stimulates intestinal epithelial cell proliferation, and ileal lactase activity is increased when porcine insulin is added to feed administered to newborn piglets (Shehadeh N. et al., Pediatr Diabetes 2001. 2:175-177; Corps A. N. and Brown K. D. J Endocrinol 1987. 113:285-90). Furthermore, milk-borne insulin affects the maturation of the pancreas and induces pancreatic amylase development in rats (Kinouchi T. et al., JPGN 2000. 30:515-521). It has been previously shown that human milk insulin concentration is significantly higher (60.23±41.05 μU/ml) compared to cows' milk (16.32±5.98 μU/ml) and that insulin is hardly detected in infant formulas. The range of insulin values in human maternal milk taken 3 to 30 days after delivery was between 6.45 to 305.65 μU/ml. In additional study, it has been further evaluated whether human insulin concentration in breast milk is affected by gestational age or postnatal age. The breast milk was analyzed for insulin levels on day 3 and 10 post partum. Human milk insulin (HMI) concentration, on either day 3 or 10 post partum, was not influenced by gestational age at delivery as well as maternal age, ethnic origin, mode of delivery, weight gain in pregnancy or maternal body mass index (BMI). The HMI concentration decreased post partum between day 3 and 10, however this decrease was only significant for mothers delivering at term (37-41 gestational age group). Thus, preterm infants are exposed to a similar concentration of insulin as term infants are (Shahadeh N et al., 2003. Arch Dis Child Fetal Neonatal Ed 88:F214-F216). Human colostrum contains higher insulin concentrations, up to about 600 μU/ml (Read L C. et al., 1984. Pediatr Res 18(2):133-139).

Enteral insulin administration may be of benefit in reducing feeding intolerance in preterm infants (Shulman R J. Arch Dis Child Fetal Neonatal Ed. 2002. 86:F131-F133), and can suppress the development of autoimmune diabetes in mice (Schatz D. A. et al., Cleve Clin J Med 1996. 63:270-4). Orally administered insulin is usually not absorbed in the gut (Larkin M. Lancet 1997. 349:1676), and the observed effects may be local and limited to the suckling period (Shehadeh N. et al., 2001, ibid). Yet, oral insulin supplementation in non-suckling mice increases insulin serum levels and has a favorable effect on serum lipid levels, suggesting a systemic effect for insulin taken orally in this population. This is in agreement with observations in adult rats, of a transcellular (but not paracellular) intestinal transport of insulin.

While no observations were made regarding long term negative effects of oral insulin, administration of oral insulin to preterm infants from 4 to 28 days of age at a concentration as high as 4 U/kg/day, increased lactase activity and may be of benefit in reducing feeding intolerance without inducing hypoglycaemia or other adverse effects (Shulman 2002, ibid). Analyzing the effect of administered insulin on mucosal mass parameters and on expression of brush border membrane (BBM) hydrolases in a suckling rat model of immature intestine also demonstrated the safety of oral insulin given in the pharmacological range of ˜10 times higher than the estimated daily intake of milk-borne insulin demonstrating the safety of oral insulin supplementation (Buts J P et al., 1997. J Pediatr Gastroenterol Nutr 25:230-2). Furthermore, this study demonstrated that insulin is able to enhance intestinal BBM enzymes prematurely especially when given in its appropriate vehicle (rat milk).

U.S. Pat. Nos. 6,365,177 and 6,399,090 to an inventor of the present invention disclose an infant formula in a powder or solution form comprising nutritional components and an insulin supplement. The insulin concentration is in the range of 10 to 1000 μU/ml solution (particularly 30-100 μU/ml solution) or 83-7,500 μU/grams of powder (particularly 250-750 μU/grams of powder), and when fed to an infant the chance of the infant to develop diabetes is reduced.

U.S. Applications Publication Nos. 20070248652 and 20060147494 disclose methods for encapsulation of active ingredients, including insulin, and formulations comprising same used to enhance the health status and growth performance of human and non-human organisms.

U.S. Pat. No. 8,026,211 discloses a method for increasing intestinal function, particularly in a subject suffering from intestinal malfunction or malnutrition, by orally and/or enterally administering a therapeutically effective amount of insulin.

Babies born weighing less than 2,500 g are considered low birth weight (LBW), and are at increased risk for serious health problems as neonates, lasting disabilities and even death. Certain LBW babies can be further classified into Very Low Birth Weight (VLBW) babies, born at less than 1,500 g, and Extremely Low Birth Weight (ELBW) babies, born at less than 1000 g. The rate of LBW neonates shows differences around the world. For example, the World Health Organization (WHO) estimated that 16.5% of births in less developed regions in the year 2000 were LBW. In contrast, around 1 of every 12 (8.3%) babies born in 2005 in the United States was born LBW. The rate of LBW babies is increasing, particularly in more developed regions such as the United States, believed to result predominantly from an increase in preterm delivery of artificially conceived multiple pregnancies.

Other then genetic background of small parents, the main reasons for low birth weight are premature birth, i.e. a baby born before 34 full-weeks from the first day of the last menstrual period, and fetal growth restriction i.e. babies that may be full-term but are underweight, also known as small-for-gestational age (SGA) or small-for-date babies. Small for gestational age (SGA) is defined as a birth weight and/or length 2 standard deviations (SDs) below the gender-specific population reference mean for gestational age. SGA can be the result of intrauterine growth retardation (IUGR), preterm birth or both.

The last weeks of normal gestation are characterized by a rapid growth of the embryo. Thus, preterm infants are exposed to extra-uterine life during a period that normally is characterized by rapid intra-uterine growth. To survive, the infant energy expenditure shifts from growth promoting actions to survival strategies, and this low-growth rate results in persistent ensemble of squealae on features including body composition, insulin sensitivity, blood pressure etc.

International (PCT) Application Publication No. WO 1998/044917 discloses a method for enhancing the growth of preterm infants involving the administration of certain long chain polyunsaturated fatty acids. It is preferred that the infants are administered an infant formula containing a combination of docohexaenoic acid and arachidonic acid.

International (PCT) Application Publication No. WO 2012/052060 discloses a method for increasing the growth velocity of a human infant, particularly underweight or preterm human infants, by the enteral administration of recombinant human bile-salt-stimulated lipase (rhBSSL).

International (PCT) Application Publication No. WO 2012/150245 discloses pharmaceutical compositions comprising an ATP-sensitive potassium (K-ATP) channel antagonist and methods for treating hyperglycaemia and/or promoting growth of a premature and/or small for gestational age infant.

There is unmeet need for feed formulae capable of assisting the preterm infant to overcome the growth retardation during the first postnatal months and reduce the extent of or occurrence of long-term adverse health effects of small for gestational age.

### SUMMARY OF THE INVENTION

The present invention relates to the use of insulin for promoting the growth and maturation of low birth weight infants, including preterm and small for gestational age (SGA) infants.

The present invention is based in part on the unexpected discovery that feeding preterm and/or SGA infants with insulin-enriched formula during the first 1-4 months from birth resulted in increase in weight, length and head circumference over the expected growth rate. Furthermore, the present invention now discloses that insulin at a concentration range resembling that of human breast colostrums or milk significantly enhanced maturation of the gastrointestinal tract, resulting in a decrease in the time required for transforming the infant to parenteral free, full enteral feeding and enabling earlier release of the low-birth weight born infant from the hospital. The present invention shows for the first time that insulin administered orally to preterm infant or otherwise underweight born infants promotes the growth of the infant at the crucial first one to six months growth period.

Thus, according to one aspect, the present invention provides a method for enhancing the growth rate of a low birth weight human infant, comprising administering insulin orally to the infant when newborn, thereby enhancing the infant growth rate over the expected rate.

According to certain embodiments, the low birth weight infant is selected from the group consisting of a preterm human infant and small for gestational age (SGA) human infant.

According to typical embodiments, enhancing the growth rate comprises a measure above the expected for the preterm or SGA infant of at least one of the infant weight, height and head circumference.

According to other embodiments, the measure of at least one of growth, height and head circumference is taken at the infant age of at least one month, two months, three months or four months. According to other embodiments, the measure is taken at the age of at least three months. According to certain typical embodiments the measure is taken at the age of six months. According to some embodiments, a plurality of these measures above expected is achieved.

According to additional embodiments, enhancing the growth rate comprises a measure above the expected for the preterm or SGA infant for gastrointestinal maturation. According to certain embodiments, the measure of the infant gastrointestinal maturation is set by the number of days required to achieve complete enteral feed. According to these embodiments, administering insulin to a low birth infant according to the teachings of the present invention reduces the number of days required to achieve complete enteral feed compared to the expected number of days.

According to additional embodiments, administering insulin to a low birth infant according to the teachings of the present invention reduces the period of hospitalization of the infant compared to the expected hospitalization period.

According to the teachings of the present invention, insulin can be administered directly, within a composition and/or within an infant formula. Any method for enteral administration known in the art can be used according to the teachings of the present invention. According to certain typical embodiments, the insulin or a composition comprising insulin is mixed with infant formula to form an insulin-enriched formula. According to certain specific embodiments, the insulin is encapsulated in an encapsulating material. Encapsulating materials are typically selected from the group consisting of polysaccharides, milk powder, whey proteins, lipids, gum Arabic and microcrystalline cellulose. Other encapsulation materials well known in the art are also encompassed within the scope of the present invention.

According to one embodiment, insulin is microencapsulated within a matrix of maltodextrin (MD) to form an insulin supplement. According to other embodiment, the matrix further comprises anti oxidant, typically vitamin C. This matrix provides the encapsulated insulin with a long term stability and resistance to exposure to high temperatures (above 42° C.) in terms of preserved activity.

Any infant formula as is known in the art can be used as a basal formula for producing the insulin-enriched formula. Typically, the infant formula is in a form of dry powder reinstated into water to form a liquid formula prior to use.

According to certain embodiments, insulin is administered as liquid insulin-enriched formula at a concentration range of from 50 microIU/ml (μIU/ml) to 600 microIU/ml (μIU/ml). According to some embodiments, the insulin-enriched formula comprises insulin at a concentration range of from 50 μIU/ml to 400 μIU/ml. According to other embodiments, the insulin-enriched formula comprises insulin at a concentration range of from 75 μIU/ml to 125 μIU/ml. According to certain typical embodiments, the insulin-enriched formula comprises 100 μIU/ml of insulin. According to other typical embodiments, the insulin-enriched formula comprises 400 μIU/ml of insulin. According to certain typical embodiments, the insulin is biologically active.

The insulin concentration range disclosed herein is significantly lower (up to 4 orders of magnitude) compared to hitherto insulin concentration known to enhance preterm gastrointestinal maturation.

Thus, according to certain embodiments, the present invention provides a method for enhancing the rate of gastrointestinal maturation of a low birth weight infant comprising orally administering to the infant when newborn a liquid composition comprising insulin at a concentration of from 50 μIU/ml to 600 μIU/ml.

According to certain typical embodiments, the enhanced maturation of the gastrointestinal maturation results in reducing the number of days required to achieve complete enteral feed of the infant. According to further embodiments, the enhanced maturation of the gastrointestinal maturation results in reducing the number of the subject hospitalization.

According to certain embodiments, the insulin is mammalian insulin selected from the group consisting of human insulin and bovine insulin. According to certain typical embodiments, the insulin is human insulin. According these embodiments, the insulin is recombinant or semi-synthetic human insulin.

The insulin or insulin enriched formula of the present invention can be administered by normal feeding, or, when this is not possible, via a nasogastric tube.

The methods of the present invention are directed to newborn infants during the initial weeks or months of life, typically during at least the first month of life, or during at least the first two month of life, and including up to about 3 months, up to 4 months, up to 5 month and up to 6 months of life or more. It is to be explicitly understood that the duration of administering insulin or insulin-enriched formula according to the teachings of the present invention can mimic the duration of breastfeeding, i.e. as long as bottle feeding is mutually desired by mother and child.

According to certain embodiments, insulin is administered as liquid insulin-enriched formula at an average daily feeding volume similar to that of breastfed infants during the initial weeks or months of life. According to certain embodiments, the insulin-enriched formula is administered together with breastfeeding. According to other embodiments, the insulin-enriched formula is administered as the sole nutrition. According to yet additional embodiments, the insulin-enriched formula is administered together with parenteral feeding. According to yet additional embodiments, the insulin enriched formula is administered in combination with additional food.

Other objects, features and advantages of the present invention will become clear from the following description and drawings.

### BRIEF DESCRIPTION OF THE FIGURES

FIG. 1 shows the mean of the observed weight compared with the mean of the expected weight at birth.

(*)—The difference between the mean observed and expected values. Effect is not significant.

(**)—The difference between the mean observed and expected values is significant at P<0.001.

FIG. 2 shows the mean of the observed length compared with the mean of the expected length at birth.

(*)—The difference between the mean observed and expected values. Effect is not significant.

(**)—The difference between the mean observed and expected values is significant at P<0.025.

### DETAILED DESCRIPTION OF THE INVENTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present invention discloses for the first time that insulin, at a concentration range of 50-600 μIU per ml of an infant formula, enhances the growth of low birth weight infants, including small for gestational age (SGA) and preterm infants when the insulin-enriched formula is administered during the first weeks or months of the infant.

Hitherto disclosed effects of oral administration of insulin to infants and/or preterm infants include an increase of intestinal function in healthy and non-healthy infants, a reduction of feeding intolerance in preterm infants and a reduction in the risk to develop diabetes at a later stage of life. The present invention discloses an additional and unexpected outcome of insulin administration to low birth weight infants. One of the major obstacles for low birth weight infant normal growth resides in their slow development during the first weeks and months of life, which has long-term effect on a variety of growth parameters. It is now shown that giving the low birth weight infant a formula mimicking breast colostrum and/or milk in terms of insulin concentration significantly enhances their growth at this critical stage, and thus may prevent the long term deleterious effect of low weight at birth. The present invention further shows that these insulin concentrations are highly effective in enhancing the maturation of low birth weight infant gastrointestinal tract, a phenomenon hitherto shown only with significantly higher insulin concentrations.

### DEFINITIONS

As used herein, the term “low birth weight” with regard to infant refers to babies born weighing less than 2,500 g, and includes babies born with very low birth weight of less than 1,500 g, and babied born with extremely low birth weight of less than 1000 g. The term further includes preterm infants and small for gestational age (SGA) infants.

As used herein, the term “preterm infant” refers to an infant born at 34 week of gestation or below. As used herein, the term includes preterm infants born at small for gestational age (SGA) and preterm infants born appropriate for gestational age (AGA).

The term “small for gestational age (SGA)” refers to babies having a birth weight and/or length at least 2 standard deviations below the mean for gestational age.

As used herein, the term “insulin” refers to a polypeptide hormone, which is naturally secreted by the islets of Langerhans and functions in the regulation of the metabolism of carbohydrates and fats, particularly the conversion of glucose to glycogen. The insulin may be native insulin (purified or synthetic or recombinant) or analogs thereof. According to certain embodiments, the term insulin refers to mammalian insulin. According to certain typical embodiments, the term insulin refers to recombinant human insulin and to analogs thereof, which is biologically active.

As used herein, the term “IU” (International Unit) refers to the biological equivalent of about 45.5 μg pure crystalline insulin (exactly 1/22 mg).

The terms “increasing” or “enhancing” (e.g. body weight) refers to at least 0.05%, 0.1%, 0.5%, 1%, 2%, 5%, 10%, 15%, 20% increase in an examined measure of the present invention including body weight, height, head circumference and gastrointestinal maturation of an infant compared to its expected value. It is to be explicitly understood that according to certain embodiments, enhancement of gastrointestinal maturation is measured by a reduction in the number of days required for transforming the low birth weight infant into a complete enteral feed. As used herein, the tern “complete enteral feed” refers to enteral feed at an amount of about 130-170, typically about 140-160 ml/Kg/day. According to other certain embodiments, enhancement of gastrointestinal maturation is assessed in accordance with the volume of gastric residuals (residuals that were left in the infant's stomach from the prior meal). The volume of the gastric residual is inversely correlated to the gastrointestinal maturation.

As used herein the term “expected measure value” with regard to body weight, height, and head circumference refers, according to certain embodiments, to the expected value obtained from the Center for Disease Control and preventions (CDC) growth reference tables (http://www.cdc.gov/growthcharts) according to the infants' gender, gestational age and percentile of birth weight (BW). According to other embodiments, the term “expected measure value” with regard to body weight, height, and head circumference refers to the expected value according to the World Heath Organization (WHO) growth standards (WHO Multicentre Growth Reference Study Group; WHO Child Growth Standards: Methods and Development. Length/height-for-age, weight-for-age, weight-for-length, weight-for-height and body mass index-for-age: Methods and development. Geneva: World Health Organization, 2006. Available at:

http://www.who.int/childgrowth/standards/technical_report/en/index.html; de Onis M, Garza C, Onyango A W, et al, editors. WHO Child Growth Standards. Acta Paediatr Suppl 2006; 450: 1-101).

According to one aspect, the present invention provides a method for enhancing the growth rate of low birth weight human infant, comprising orally administering insulin to the infant when newborn thereby enhancing the infant growth rate over the expected rate.

According to certain embodiments, insulin is administered within a liquid formula forming insulin-enriched liquid formula. According to some embodiments, the insulin-enriched formula comprises insulin at a concentration range of from 50 μIU/ml to 600 μIU/ml. According to some embodiments, the insulin-enriched formula comprises insulin at a concentration range of from 50 μIU/ml to 500 μIU/ml. According to additional embodiments, the insulin-enriched formula comprises insulin at a concentration range of from 50 μIU/ml to 400 μIU/ml. According to other embodiments, the insulin-enriched formula comprises insulin at a concentration range of from 75 μIU/ml to 125 μIU/ml. According to certain typical embodiments, the insulin-enriched formula comprises 100 μIU/ml of insulin. According to other typical embodiments, the insulin-enriched formula comprises 400 μIU/ml of insulin. According to certain typically embodiments, the insulin is biologically active.

According to certain embodiments, the insulin is mammalian insulin selected from the group consisting of human insulin and bovine insulin. According to certain typical embodiments, the insulin is human insulin. According these embodiments, the insulin is recombinant or semi-synthetic human insulin.

Insulin concentration in human colostrums after full term delivery was found to be in the range of from about 400 μU/ml to 600 μUml (Ontsouka C E et al., 2004. Somestic. Animal Endocrinol 16:155-175; Read 1984, ibid). Insulin concentration milk after full-term delivery was found to be lower, at an average of about 60 μIU/ml. Similar values were measured in breast milk of mothers of preterm infant, regardless of the gestational age (Shehadeh N. et al. 2003. ibid; Shehadeh N. et al. 2006 J Pediatr Gastroenterol Nutr 43:276-281).

Insulin has been suggested as one of the trophic factors present in colostrum of several mammalian species, including human and pigs. Its concentration in human and pig colostrum is 3-30 fold greater than that in serum (human serum concentration being in the range of 7-24 μIU/ml) and decreases in parallel to decrease in the colostrum trophic activity. Shulman (1990, ibid) demonstrated that intestine ileal mass and lactase activity increased in newborn miniature pigs in response to oral administration of 85 mU/ml of insulin. The increase in the ileal mass was significant enough to affect the total small intestinal mass, which was found to be higher in groups treated with insulin compared to non-treated groups. In this and other studies it has been shown that oral intake of insulin in the concentrations examined does not affect the blood sugar or insulin level, indicating that insulin is not absorbed systemically to any significant degree. Shulman (2002, ibid) showed that enteral administration of insulin at a high concentration of 4 U/ml to preterm human infants enhances gastrointestinal function, as measured by increased lactase activity.

Any method as is known in the art for oral delivery of a compound to an infant can be used according to the teachings of the present invention. According to certain embodiments, insulin is administered within a semi-solid insulin-enriched infant formula. According to other embodiments, insulin is administered as liquid insulin-enriched infant formula. According to some embodiments, the infant formula is administered at an average daily feeding volume similar to that of breastfed infants during the initial weeks or months of life.

Calculations of the optimal formula quantities to be administered to newborn to 6 months old infants are based on energy and protein consumption as observed in healthy infants receiving breast milk as the sole nutrition. Assuming energy content of the formula to be 60-75 kcal/100 ml (according to the minimum permitted protein content in the EU) recommended formula consumption is between 115 to 215 ml/Kg/day (Report of the Scientific Committee on Food on the Revision of Essential Requirements of Infant Formulae and Follow-on Formulae, May 2003).

As is shown in the Examples section hereinbelow, in concomitant with the previous results, no adverse events were observed in all the infants who participated in the studies up to 6 months of follow up. The addition of insulin to infant formula did not cause hypoglycemia, and did not stimulate the production of anti-insulin antibodies.

According to typical embodiments, enhancing the growth rate comprises a measure above the expected for preterm and/or SGA infant of at least one of the infant weight, height and head circumference at the age of six months. According to some embodiments, a plurality of these measures above expected is achieved.

Relative to the expected growth according to the CDC growth tables, infants fed with insulin showed enhanced growth in weight, length and head circumference. Relative to the expected growth at 6 months, the strongest effect was on weight (18.1%) followed by head circumference (17.6%) and then by length (8.1%).

The data of the study presented hereinbelow were compared to expected values taken from published tables. The data presented in these tables may differ from on-site measured growth rates, particularly due to differences in populations (the CDC tables refer to U.S. population while the study was performed in Israel). In addition, the expected growth taken from the CDC published tables are not specific with respect to gestation and plurality of births. Data obtained from a control study may thus provide even more significant effect of insulin administration according to the teachings of the present invention.

Unexpectedly, the present invention further shows that low insulin concentration in the range of up to 400 μU/ml, which resembles the insulin concentration of human breast colostrums and milk, is highly effective in enhancing the gastrointestinal tract maturation of preterm and SGA infants, a phenomenon hitherto demonstrated with significantly higher insulin concentrations (e.g. Shulman 2002, ibid).

As presented in the Example section hereinbelow, administration of insulin at 400 μU/ml resulted in reduction of the days required for transforming the treated preterm infant to a complete enteral feeding. This outcome is of high significance as it enables early discharge of the preterm infant from the intensive care unit and even from the hospital to home care, and thus reducing the risk of contamination. The early infant discharge is also of significant from en economical point of view, significantly reducing the cost involved in preterm or otherwise low for birth infant hospitalization.

Insulin is barely detectable in currently available infant formulae (Shehadeh N. 2001. Acta Paediatr. 90:93-95). Infant formulae are typically produced from bovine milk, in which the insulin concentration is low (typically less than one third) compared to its concentration in human milk. In addition, the harsh conditions involved in the formulae production result in the loss of insulin, particularly of biologically active insulin.

Thus, according to certain currently typical embodiments, the insulin is encapsulated within encapsulating material providing stability to the insulin. As used herein, the term “insulin stability” refers to maintaining at least 60%, 70%, 80%, 85%, 90% 95% or 100% of the insulin initial activity. Methods of encapsulating insulin are known in the art. Examples of such methods are provided in International Patent Applications Publication Nos. WO 2004/112494 and WO 2005/115473, assigned to the Applicant of the present invention.

In the food and pharmaceutical industries, for example, microencapsulation is used to stabilize the core material, to control the timing and rate of the release of the core material and to separate and prevent chemical interaction between reactive or incompatible components of a multicomponent formulation. Thus, microencapsulation makes it possible to protect sensitive bioactive agents, to ensure against activity loss and to mask or preserve flavors and aromas. Encapsulation may be used to preserve biological activity of bioactive ingredient, such as growth promoting agents against any of the following or similarly destructive factors: adverse temperature, pressure, humidity, pH, osmotic concentration, ionic concentration, chemical degradation, presence of metals, surfactants and chelators, radiation (including but not limited to UV, IR, visible light), enzymatic and microbial degradation and combinations thereof.

Release of the encapsulated bioactive ingredient may occur spontaneously in the digestive tract, or may be the result of environmental events.

According to certain embodiments, a protective layer surrounding or incorporating the insulin is specifically designed to degrade, or undergo controlled release as a response to exposure to the change in environmental condition. The change in the environmental condition can be time, temperature, moisture content, pressure, or pH, ionic strength, enzymatic activity, or a combination thereof. According to other embodiments, the insulin is encapsulated in a material designed to protect it from digestion in the digestive system of the infant and to release the insulin only as a response to an increase in pH. The insulin may be further encapsulated with another encapsulating material, designed to protect the core from increased temperature. The skilled artisan in the art, would recognize that the order of environmental triggers releasing the active compound is not rigid and depends on the environmental conditions of manufacturing, environmental conditions of integration into food products, environmental conditions of storage after integration onto food products, desired delivery location within the gastrointestinal system, timing and physiological activity desired.

Any factor that may affect the entrapment of insulin in a biodegradable matrix and thereby affect its initial loading, subsequent release, or a combination thereof, may be utilized. Such factors may comprise inter-alia, the initial solvent concentration, its molecular size and polarity, the temperature and pressure under which the solvent is removed, molecular weight number (MWn) average of the biodegradable matrix, and its polydispersity index. When the biodegradable matrix is a polymer, the size and polarity of the insulin, the monomer ratio and distribution along the copolymer's chain, or a combination thereof may be also considered. In addition, D/L ratio within each monomer of a biodegradable polymer will affect release rates. The term D/L ratio refers to the ratio of monomer molecules that affect the direction (D-right, L-left), in which a cross-polarized lens will be rotated when observing a single optically active monomer like lactic acid. Since most mammals have D-specific enzymes, that ratio will affect the digestion rate of the biodegradable biopolymer, affecting its molecular weight and consequently its viscosity, thereby affecting release rate of the entrapped insulin.

Various materials may be used as the encapsulated material as described, for example, in WO 2004/112494 and WO 2005/115473 cited above. According to certain currently preferred embodiments, insulin is microencapsulated within a matrix of maltodextrin (MD) and vitamin C as described in WO 2005/115473.

The encapsulated insulin can be mixed with any infant formula as is known in the art. The encapsulation can also protect the insulin in a manner that, when a liquid formula containing the encapsulated insulin in consumed by an SGA infant, the insulin is protected, at least partially, during its passage through the newborn gastrointestinal track or stomach such that sufficient amount of insulin is still active to exert its growth-enhancing activity as described herein.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

### Clinical Study: Effect of Insulin on Growth Parameters of Low Birth Weight Infants

### Study Population

The effect of insulin enriched formula on low birth weight full term infant was examined. Inclusion criteria for participating in the study included: healthy infants 34-42 weeks gestation (“term born infants”); birth weight above 1,650 g, typically between 1750 gram and 2500 gram; no major congenital malformation or any other illness sign; chronological age under 240 hours; mother is not willing to breastfeed the baby during the study period and signed an informed consent. Infant with a high index of suspect for infection before enrolment, maternal diabetes, and medications given until day 1 of the study were excluded.

Observed results regarding weight, length, and head circumference were compared to expected values which were obtained from the CDC growth reference tables (ibid) according to the infants' gender, gestational age and percentile of birth weight (BW). Interpolation or extrapolation was used to determine the percentile and the expected gain of each infant. The expected values are specific to the percentile measure at birth. The percentile of each BW was evaluated according to the gender, gestational age and multiplicity (singleton or twin) using published tables for Israel (Dolberg S. et al., IMAJ, 7, 2005, 311-314). No such data were available with respect to the percentiles of length and of head circumference. It was therefore assumed that the BW percentile of each infant is applicable to each of the other two growth measures.

The study was conducted in accordance with the guidelines of the Declaration of Helsinki on Biomedical research involving human subjects (South Africa revision 1996) and in accordance with the GCP ICH guidelines.

### Study Design

Phase I, multi center, non-randomized, open label, single group study to assess the safety of insulin enriched formula on low birth weight infants was conducted in Rabin Medical Center and Laniado Hospital, Israel. The possible effect of the insulin enriched formula on growth of low birth weight infants, born on term (at least 34 weeks of gestation) was also examined in this study. Parents of eligible infants, who met the study criteria, were invited to participate in the study following signing on Informed Consent Form (ICF). From study day 1, the infants received the insulin enriched infant formula (Materna, Maabarot, Israel). They were fed with the formula for the first 4 months without the addition of complementary feed. The infants' gender, family origin, gestational age at birth, maternal medications, maternal age, chronic diseases in the family and the infant's condition upon enrollment were recorded.

Standard care of infants in the nursery did not change during the observation period. At study day 1 and 2, and at 1, 4, 8, 12, 16 and 24 weeks the parents of the infant were asked to come to the neonatal ward for follow up examinations. During the study, the infant's weight, length, head circumference, glucose blood levels, blood count, blood fat, blood chemistry, blood amino acids, insulin antibodies, and serum levels were recorder, as well as food consumption, vomiting, regurgitation and stools frequency were measured and assessed.

The possible effect of the insulin enriched formula on growth was evaluated with respect to gain in weight, length, and head circumference. Assuming that the largest effect should be noted at 6 months of age (the last follow-up), tests of significance were applied to the difference between the observed and expected growth at 6 months. In addition, in order to consider differences in growth during the treatment (first 4 months), and after the treatment (next 2 months), similar tests were applied to the growth during the first 4 months.

Infants were to be withdrawn from the study if any serious adverse event or any deterioration in the infant clinical condition had occurred. They were followed up on possible side effects and efficacy measures until 6 months of age. No serious adverse events were observed. A multicenter, double blind, randomized study for studying the effect of the insulin enriched infant formula on the growth of low weight infants was planned to take place in Israel in 2005 (headed by Prof Sirota, Schneider Hospital). Due to low recruitment rate (only 8 premature infants), the study was terminated. However, no adverse events (including no hypoglycemia) were recorded during the time the infants were feed with the insulin-enriched formula.

The study product InsuMeal is a dry powder, composed of insulin (Actrapid® HM (ge), biosynthetic Human Insulin, Solution for Injection. Concentration of 100 IU/ml, manufactured by Novo Nordisk), microencapsulated within a matrix of Maltodextrin 18 (MD Pharmagrade, corn starch polysaccharide, manufacture by Cargill Ltd.) and Vitamin C (Pharma-grade) that is mixed with a Premium 1—baby formula, manufactured by Materna Ltd. The microencapsulation process enables insulin bioactivity protection until its immersion and consumption within infant formula. InsuMeal is added into 60 ml boiled lukewarm water, and then the formula has the concentration of 90 μU Insulin/ml formula; mimicking a representative physiologic insulin levels in breast milk.

### Data

The data presented in Tables 1-4 hereinbelow were obtained from 11 infants of whom only 10 met the inclusion criteria. The birth weight of one (no. 28) was 1,550 gr., while the minimum value for inclusion was set at 1,750 gr. That infant was excluded from the inferential analyses.

In addition, another infant (no. 22) was excluded from the inferential analyses related to head circumference. That exclusion as explained below is based on the fact that the head circumference measured at birth is most likely an outlier.

The measurements made on these infants are presented in the tables, but excluded from the relevant inferential analyses (means, confidence intervals and tests of significance). Table 1 presents the growth measures (weight, length and head circumference) for all infant recruited to the study.

TABLE 1
Growth measures by age

| | | | | | | | | | | Circumference | | |
|---|---|---|---|---|---|---|---|---|---|---|---|---|
| | | | | Weight (Kg.) by | | | Length (cm.) by | | | (cm.) | | |
| | | Single/ | Gest. | age (month) | | | age (month) | | | by age (month) | | |
| ID | Gen. | Twin | age | 0 | 4 | 6 | 0 | 4 | 6 | 0 | 4 | 6 |
| 21 | M | T | 37 | 2.270 | 5.660 | 7.075 | 46.0 | 60.0 | 64.0 | 32.0 | 39.5 | 42.3 |
| 22 | F | T | 37 | 2.070 | 5.380 | 6.070 | 46.0 | 60.0 | 61.0 | 38.0 | 39.5 | 41.0 |
| 23 | M | S | 38 | 2.180 | 5.500 | 6.550 | 45.0 | 54.0 | 63.0 | 32.0 | 41.0 | 43.0 |
| 24 | F | T | 38 | 2.090 | 4.950 | 5.950 | 45.0 | 59.0 | 61.0 | 31.0 | 39.0 | 41.0 |
| 25 | F | T | 38 | 2.100 | 5.090 | 5.920 | 44.0 | 54.0 | 63.0 | 31.5 | 39.0 | 41.0 |
| 26 | M | S | 34 | 1.910 | 5.330 | 6.800 | 43.5 | 56.0 | 64.0 | 30.5 | 40.0 | 42.0 |
| 27 | M | S | 35 | 2.170 | 6.580 | 7.700 | 47.5 | 65.0 | 66.0 | 32.3 | 40.0 | 42.5 |
| 28 | M | S | 36 | 1.550 | 4.000 | 4.800 | 40.0 | 51.0 | 54.0 | 29.0 | 35.5 | 37.5 |
| 81 | F | T | 37 | 1.838 | 5.280 | 5.376 | 43.0 | 58.5 | 60.5 | 30.0 | 38.0 | 39.5 |
| 82 | F | T | 37 | 2.020 | 4.286 | 6.735 | 47.0 | 54.6 | 63.5 | 31.0 | 37.3 | 40.3 |
| 83 | F | S | 35 | 2.349 | 5.878 | 6.395 | 49.0 | 62.5 | 68.2 | 33.2 | 40.0 | 42.0 |

### Methods of Analyses

The primary endpoint with respect to efficacy was set as growth in terms of Z score (corrected for gestational age) during the first 6 months. However, the treatment effect was also evaluated for change in Z scores during the first 4 months, and during the period from 4 to 6 months.

Under normal distribution the Z value represents the distance (of, e.g., the weight, from the mean) in standard deviation (SD) units. However, the actual distribution of infants' weights has a longer left tail than that of the normal distribution. In order to correct the Z values associated with low percentiles, the CDC published formulas and parameters (L, M, S) that enable calculation of the correct Z value (Kuczmarski R J et al. 2000. CDC growth charts for the United States: Methods and development. National Center for Health Statistics. Vital Health Stat 11 (246). 2002). These published values are related to term newborns. Although the estimated Z values are not applicable as predictor measures, they are useful for comparing two groups of treatments with respect to the gain in weight.

The Z score values were evaluated for each infant according to the gender and the age corrected for short gestation. These parameters are presented in the CDC tables for whole month of chronological age. Accordingly, the age was corrected for a range of weeks. For example, the Z value for an infant at 6 months (chronological) age born after 38 weeks gestation was evaluated using parameters presented in the CDC tables for term babies aged 5-5.99 months. Table 2 presents for each infant the corrected Z score by chronological age. The mean and its 95% confidence interval (CI) of Z are presented for each age at the bottom of the table. Infant No. 28 was excluded from the means and CI of all measures as he did not meet the inclusion criterion regarding birth weight. Infant No. 22 was excluded with respect to means and CIs of head circumference. Exclusion of No. 22 is related to the extremely large Z score of the head circumference at birth (Z=1.82). The Z value seems to be an outlier, as it is far larger than the range of Z values (−0.98 to −3.42) of the other 10 infants. It is also extremely rare even with regards to the general population as only 3.4% in the general population are expected to have that or a larger head circumference at birth.

Statistical analyses were applied to the Z scores assuming t-distribution. However for ease of interpretation the growth measures are presented also as percentiles (Table 3).

TABLE 2
Growth measures in terms of Z score corrected for short gestation, by month of
chronological age

| | Weight | | | Length | | | Circumference | | |
|---|---|---|---|---|---|---|---|---|---|
| | | 4 | 6 | | 4 | 6 | | 4 | 6 |
| ID | Birth | months | months | Birth | months | months | Birth | months | months |
| 21 | −1.993 | −0.950 | −0.637 | −1.486 | −0.860 | −0.855 | −1.699 | −1.506 | −0.812 |
| 22 | −2.457 | −0.673 | −1.140 | −1.441 | −0.187 | −1.323 | 1.820 | −0.745 | −0.863 |
| 23 | −2.108 | −1.167 | −1.283 | −1.854 | −3.774 | −1.283 | −1.699 | −0.525 | −0.299 |
| 24 | −2.425 | −1.290 | −1.300 | −1.928 | −0.594 | −1.323 | −2.597 | −1.148 | −0.863 |
| 25 | −2.409 | −1.088 | −0.207 | −2.44 | −2.648 | −0.549 | −2.206 | −1.148 | −0.863 |
| 26 | −2.430 | −0.405 | −0.288 | −2.401 | −1.542 | −0.086 | −2.203 | −0.425 | −0.473 |
| 27 | −2.121 | 1.205 | 0.746 | −0.931 | 1.985 | 0.678 | −1.589 | −0.425 | −0.116 |
| 28 | −2.804 | −3.411 | −3.850 | −3.657 | −3.894 | −6.035 | −2.628 | −3.822 | −4.175 |
| 81 | −2.819 | −0.815 | −2.085 | −2.980 | −0.798 | −1.515 | −3.424 | −1.995 | −2.146 |
| 82 | −2.537 | −2.272 | −0.288 | −0.978 | −2.400 | −0.354 | −2.597 | −2.622 | −1.448 |
| 83 | −1.993 | 0.970 | −0.055 | −0.117 | 1.783 | 2.269 | −0.977 | 0.440 | 0.488 |
| Mean | −2.33 | −0.65 | −0.65 | −1.66 | −0.90 | −0.43 | −2.11 | −1.04 | −0.72 |
| (CI) | (−2.52; | (−1.39; | (−1.23; | (−2.62; | (−2.12; | (−1.27; | (−2.66; | (−1.75; −0.33) | (−1.31; −0.14) |
| | −2.14) | 0.10) | −0.08) | −1.05) | 0.41) | −0.40) | −1.56) | | |

TABLE 3
Growth measures in terms of percentile corrected for short gestation, by month of
chronological age

| | Weight | | | Length | | | Circumference | | |
|---|---|---|---|---|---|---|---|---|---|
| | | | 6 | | | 6 | | 4 | 6 |
| ID | Birth | 4 months | months | Birth | 4 months | months | Birth | months | months |
| 21 | 2.3 | 17.1 | 26.2 | 7.5 | 19.5 | 19.6 | 4.5 | 6.6 | 20.8 |
| 22 | 0.7 | 25.1 | 12.7 | 7.5 | 42.6 | 9.3 | 96.6 | 22.8 | 19.4 |
| 23 | 1.8 | 12.2 | 10.0 | 2.7 | 0.0 | 10.0 | 4.5 | 30.0 | 38.2 |
| 24 | 0.8 | 9.9 | 9.7 | 2.7 | 27.6 | 9.3 | 0.5 | 12.5 | 19.4 |
| 25 | 0.8 | 13.8 | 41.8 | 0.7 | 0.4 | 29.2 | 1.4 | 12.5 | 19.4 |
| 26 | 0.8 | 34.3 | 38.7 | 0.3 | 6.2 | 46.6 | 1.4 | 33.5 | 31.8 |
| 27 | 1.7 | 88.6 | 77.2 | 22.5 | 97.6 | 75.1 | 5.6 | 33.5 | 45.4 |
| 28 | 0.3 | <0.1 | <0.1 | <0.1 | <0.1 | 0.0 | 0.4 | <0.1 | <0.1 |
| 81 | 0.2 | 20.8 | 1.9 | 0.1 | 21.2 | 6.5 | <0.1 | 2.3 | 1.6 |
| 82 | 0.6 | 1.2 | 38.7 | 16.4 | 0.8 | 36.2 | 0.5 | 0.4 | 7.4 |
| 83 | 2.3 | 83.4 | 47.8 | 45.4 | 96.3 | 98.8 | 16.4 | 67.0 | 68.7 |
| | 1.0 | 25.8 | 25.8 | 4.8 | 18.4 | 33.4 | 1.7 | 14.9 | 23.5 |
| | (0.6; | (8.2; | (10.9; | (0.4; | (1.7; | (10.2; | (0.4; | (4.0; | (9.5; |
| | 1.6) | 54.0) | 46.8) | 14.7) | 65.9) | 34.5) | 5.9) | 37.1) | 44.4) |

Mean and 95% confidence interval (CI) of the Z score were evaluated for each of the three measures by age (birth, 4 months and 6 months).

Analyses were performed under the null hypothesis that the natural growth of each infant is expected to remain on the same percentile; namely, the Z score is expected to be stable during growth. Thus, the effect of the supplemented feed was evaluated as the difference between the Z score at 6 months and that at birth. The mean (and 95% CI) of the difference in the Z scores were evaluated and tested for significance at 5% level using two-tail t-test. The growth during each of the other two periods was similarly evaluated and tested for significance by the paired differences in Z scores. Table 4 presents the mean difference in the Z score and its 95% confidence interval for each of the three measures.

TABLE 4
Significance value, mean and 95% confidence interval of growth in terms of Z
scores (corrected for gestation) in each period.

| Period | | | | | | |
|---|---|---|---|---|---|---|
| (months) | Weight | P | Length | P | Circumference | P |
| 0 to 4 | 1.68 (1.01; 2.35) | <0.0001 | 0.75 (−0.35; 1.86) | 0.158 | 0.71 (−0.21; 1.62) | 0.115 |
| 0 to 6 | 1.68 (1.17; 2.18) | <0.0001 | 1.22 (0.64; 1.80) | 0.001 | 0.98 (0.04; 1.92) | 0.043 |
| 4 to 6 | −0.01 (−0.68; 0.67) | NS | 0.47 (−0.57; 1.51) | NS | 0.27 (−0.02; 0.56) | 0.064 |

### Results

Side Effects

None of the primary side effects including hypoglycemia or insulin antibodies were observed in any of the infants. Hematological tests including complete blood count and white blood cells were normal. General chemistry including glucose levels, albumin, globulin, SGot, GGT, ALP, Na, K, Ca, UA, creatinine, amylase, amino acids, anti-insulin antibodies, and lipid profile all were within the normal range. Irritability and sleeping history, allergic reaction, or gastrointestinal infection were assessed and no abnormality was noted. A single possible secondary side effect was seen in one infant with respect to triglycerides (328 mg/dL).

Weight

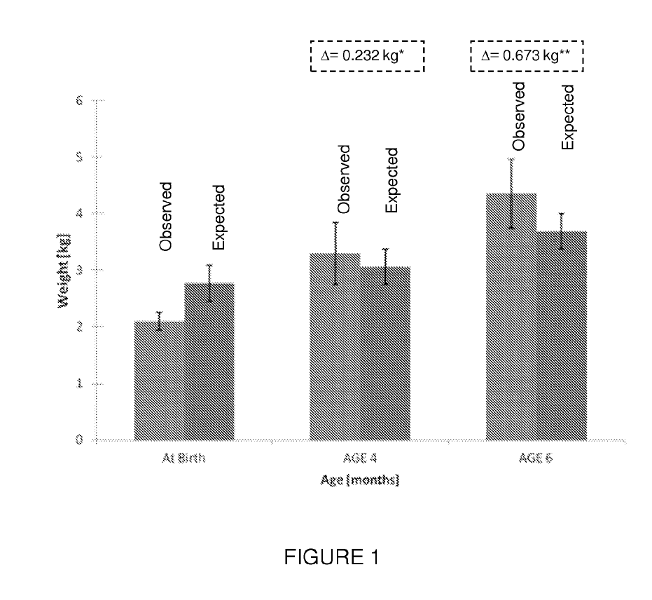

The weight of all the 10 infants analyzed was larger than expected. At the age of 6 months, the mean and 95% confidence limits of the difference (in Kg) between the observed and expected values were 0.67 (0.37; 0.96). Results are highly significant, P<0.001 (FIG. 1).

Results related to gain in weight during the first 4 months show that the growth of 6 out of the 10 infants was larger than expected. The mean and 95% confidence interval of the difference between the observed and expected net gain (in Kg.) at 4 months were 0.23 (−0.12; 0.59). The effect at 4 months is not significant (FIG. 1).

Length

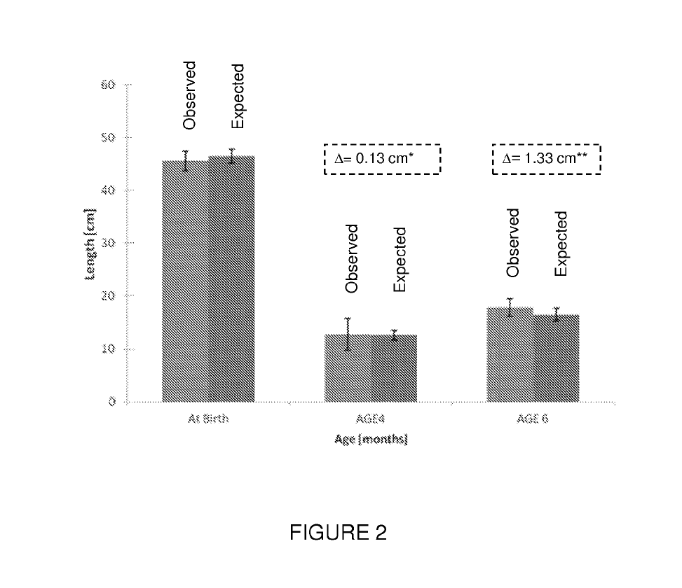

Ten out of eleven infants were included in the length comparison. At the age of 6 months, the mean and 95% confidence limits of the paired difference between observed and expected length growth (in cm) were: 1.33 (0.21; 2.45), P<0.025 (FIG. 2).

Head Circumference

Ten out of eleven infants were included in the head circumference comparison. The mean and 95% confidence limits of the difference between the observed and expected circumference growth (in cm) at the age of 6 months were: 0.85 (−0.65; 2.35). The difference is not significant.

In order to consider the possibility that the results are heavily affected by over-corrected age for gestational age, analyses were also applied to the data assuming full term gestation. Table 5 presents the Z scores of each growth measure. Table 6 presents significance of the change in Z score at each period as well as the means and 95% CI of the changes. Results are highly significant (P<0.001) for 6 months growth in weight and in head circumference. During 6 months the mean weight moved from percentile 1.0 to percentile 25.8; during that time mean growth in circumference was from percentile 1.7 to percentile 7.8. The change in length, although not quite significant (P<0.11), increased from the 4.9 to the 9.2 percentile

Thus, the results clearly indicate that the enhanced growth of low the infants is not related to overcorrection of age.

TABLE 5
Growth measures in terms of Z score uncorrected for short gestation, by month of
chronological age

| | Weight | | | Length | | | Circumference | | |
|---|---|---|---|---|---|---|---|---|---|
| | | 4 | 6 | | 4 | 6 | | 4 | 6 |
| ID | Birth | months | months | Birth | months | months | Birth | months | months |
| 21 | −1.993 | −1.762 | −1.233 | −1.486 | −1.800 | −1.575 | −1.699 | −2.184 | −1.306 |
| 22** | −2.457 | −1.421 | −1.723 | −1.441 | −1.010 | −1.932 | 1.820 | −1.489 | −1.393 |
| 23 | −2.108 | −1.987 | −1.900 | −1.854 | −4.962 | −2.022 | −1.699 | −1.172 | −0.781 |
| 24 | −2.425 | −2.031 | −1.887 | −1.928 | −1.407 | −1.932 | −2.597 | −1.921 | −1.393 |
| 25 | −2.409 | −1.830 | −1.927 | −2.440 | −3.382 | −1.185 | −2.206 | −1.921 | −1.393 |
| 26 | −2.430 | −2.232 | −1.576 | −2.401 | −3.816 | −1.575 | −2.203 | −1.852 | −1.531 |
| 27 | −2.121 | −0.556 | −0.503 | −0.931 | 0.303 | −0.731 | −1.589 | −1.852 | −1.157 |
| 28* | −2.804 | −4.393 | −4.598 | −3.657 | −6.887 | −6.984 | −2.628 | −4.669 | −4.846 |
| 81 | −2.819 | −1.561 | −2.688 | −2.980 | −1.605 | −2.118 | −3.424 | −2.826 | −2.717 |
| 82 | −2.537 | −3.018 | −0.868 | −0.978 | −3.146 | −0.996 | −2.597 | −3.495 | −1.998 |
| 83 | −1.993 | −0.739 | −1.299 | −0.117 | −0.015 | 0.803 | −0.977 | −1.070 | −0.564 |
| Mean | −2.33 | −0.65 | −0.65 | −1.66 | −2.08 | −1.33 | −2.11 | −2.03 | −1.43 |
| (CI) | (−2.52; | (−1.39; | (−1.23; | (−2.26; | (−3.30; −1.05) | (−1.96; −0.70) | (−2.66; −1.56) | (−2.61; −1.45) | (−1.92; −0.89) |
| | −2.14) | −0.08) | −0.08) | −1.05) | | | | | |

*Excluded from mean and CI evaluation.
**Excluded from mean and CI evaluation related to head circumference.

TABLE 6
Significance value, mean and 95% confidence interval of growth in terms of Z
scores (uncorrected for gestation) in each period.

| | Weight | P | Length | P | Circumference | P |
|---|---|---|---|---|---|---|
| 4-0 | 0.62 (0.16; 1.08) | 0.014 | −0.43 (−1.48; 0.62) | NS | 0.08 (−0.34; 0.50) | NS |
| 6-0 | 0.77 (0.40; 1.13) | 0.001 | 0.33 (−0.09; 0.75) | 0.109 | 0.68 (0.48; 0.89) | <0.0001 |
| 6-4 | 0.15 (0.47; 0.78) | NS | 0.76 (−0.32; 1.84) | NS | 0.61 (0.30; 0.91) | 0.002 |

In conclusion, the growth during 6 months is significant for each of the three measures. On the average the growth in weight during 6 months moved the infants from the 1.0 to the 25.8 percentile. The mean growth in length was from 4.9 to 33.4 percentile. The mean growth in head circumference was from the 1.7 to the 23.4 percentile.

### Example 2

### Clinical Study: Effect of Insulin on Growth Parameters of Low Birth Weight Preterm Infants

A Multi-center, two arms, randomized, double-blinded placebo controlled study was conducted to evaluate the effect of insulin-enriched infant formula on preterm infants. In this study, the population of infants included also babies having extremely low birth weight (inclusion criteria included babies weighing over 750 grams). The insulin was given at a concentration range which is mimicking colostrums concentration (at 400 μU/ml. The study primary goal was to determine whether insulin supplement to the basic preterm oral formula enhances gastrointestinal maturation. The gastrointestinal maturation was evaluated by the ability of the premature infants to achieve complete enteral feeds (150-160 ml/kg/day).

### Study Design

Parents of eligible preterm infants who meet the study criteria described hereinbelow were invited to participate in the study following signing of an Informed Consent Form (ICF).

The infants were hospitalized in the neonatal hospital ward in the same manner as preterm infants whose parents elected not to participate in the study. The infants were randomly assigned to one of two treatment groups: the study product—InsuMeal™ as a test group and Placebo supplement as a control group. The parents, the medical team treating the infants and the study monitor were blinded to the treatment arm. From study day 1, the infants in the test group received the InsuMeal™ additive mixed with the ready to feed (RTF) preterm Materna formula and the control group received a placebo supplement mixed with the same RTF preterm Materna formula.

The infants' gender, family origin, gestational age at birth, maternal medications, maternal age, chronic diseases in the family and the infant's condition, weight, length and head circumference upon enrollment were recorded.

During the study, the infant's weight, food intake, food consumption, glucose blood levels, total parenteral nutrition (TPN) received, gastric residuals and stool data (frequency and consistency), vomiting, and regurgitation were assessed daily from the study start point throughout day 28 or discharge, if achieved prior to day 28. On study day 28 or discharge day, as well as on the 3 month follow up visit, 2.5 ml blood were drawn to further asses the glucose blood levels, complete blood count, lipid profile, blood general chemistry, blood amino acids, and anti-insulin antibodies. The infant length and head circumference were measured on day 1, 7, 14, 21 and 28 as well as at the 3 at 6 months visit. A physical examination was completed as well.

33 preterm infants, aged up to 7 days old, born between 26-33 weeks of pregnancy, weighing over 750 grams, who are free from high index suspicion for infection showing stable condition were enrolled into this study.

Randomization was carried by block design. Each site received a randomly selected block of size 4 in which consecutive infant ID numbers was allocated to either one of the two groups (InsuMeal/placebo). Once enrollment of the four infants block is completed, the site received an additional block.

One subject was withdrawn due to health complications; 32 subjects were included in the analyses, 15 males and 17 females aged between 1-7 days old.

### Inclusion Criteria

The following criteria had to meet in order to be included in the study:
1. Pre-term infants born after 26-33 weeks gestation. Gestational age matching (±2 weeks) between maternal dates and early antenatal ultrasound.
2. Birth weight≧750 gr.
3. Postnatal age≦7 days.
4. Fraction of inspired oxygen≦0.60 at enrollment.
5. The infant is in a cardiovascular stable condition.
6. No breast feeding after study day 1.
7. No heart and chest compression or any resuscitation drugs given to the infant during delivery
8. Informed consent form signed by parents or legal guardian.

### Exclusion Criteria

Infants who meet one or more of the following criteria were excluded from the study:
1. Pre-term infants age <26 or >33 weeks gestation. Gestational age matching (±2 weeks) between maternal dates and early antenatal ultrasound.
2. Birth weight <750 grams.
3. Postnatal age >7 days.
4. Fraction of inspired oxygen >0.60 at enrollment.
5. The infant is in cardiovascular instability
6. Breast feeding after study day 1.
7. Major congenital malformation—Infants with genetic metabolic or endocrine disorder diagnosed before enrollment (including disorders diagnosed after enrollment but are known to be congenital).

8. High index of suspicion of infection before enrollment.

Complete Oral Feeding.
9. Infant developing necrotizing enterocolitis or is suspected of having necrotizing enterocolitis.
10. Maternal diabetes.
11. The infant is treated with Insulin
12. NPO, nothing per os for any reason at the study entry.
13. Heart and chest compression or any resuscitation drugs given to the infant during delivery
14. participation in another clinical study

### Trial Products

The study products included InsuMeal™ and placebo. InsuMeal™ is an insulin based additive intended to be mixed with 90 ml RTF preterm infant formula (manufacturer: Nestle Germany, importer: Materna Laboratories, Israel. Once InsuMeal™ is added to the formula, the formula has the concentration of 400 μU Insulin/ml formula; mimicking the insulin levels in breast milk.

The InsuMeal™ additive comprises three components:

(a) Human insulin, which is a large protein (5,800 Daltons) composed of two polypeptide chains joined by two disulfide bonds. Insulin is a natural health promoting component present in mammalian milks, at concentrations of nanogram per milliliter. Insulin is also classified as a peptide hormone when injected in therapeutic dosages by individuals diagnosed with Diabetes Mellitus.

(b) Maltodextrin which is a mixture of polysaccharides, produced by the partial hydrolysis of starch. Maltodextrin is commonly used as a component in infant's formula and dissolves immediately in liquid.

(c) Vitamin C or L-ascorbic acid is an essential nutrient for humans. In living organisms, ascorbate is an anti-oxidant, since it protects the body against oxidative stress, and is a cofactor in several vital enzymatic reactions. In InsuMeal™ formulation vitamin C is used as an indicator for insulin oxidation.

The placebo consists of Maltodextrin and vitamin C.

The InsuMeal™ was used according to study definitions and feeding protocol only. Formula administration will began at study day 1 and continued throughout the following 28 days or discharge day if achieved prior to day 28.

The InsuMeal™ was kept at room temperature and out of reach of children. InsuMeal™ has no contraindications.

### Product Administration

The content of the study sachet (InsuMeal/placebo), marked per infant, was added by the neonatal intensive care unit (NICU) nurses into a 90 ml glass bottle of RTF premature infants Materna formula, right before each meal. Once added, the bottle was closed and shaken well to insure the additive has completely dissolved. The infants received a new 90 ml glass bottle+additive per meal. Standard feeding bottles and nipples were used. Once the infant finished the meal, the left over formula was kept in the glass bottle for 24 hours for lab use only. If the infant was found to be stable and well, the bottle was discarded, otherwise the sponsor was informed and collected the bottle for examination.

### Feeding Protocol

Starting at birth, the preterm infant received parenteral nutrition (PN). PN could be stopped before the newborn reaches complete enteral feeds (150 cc/kg/day). For the first 3 consecutive days that the infant was fed (could be prior to the study, he received at least 10 ml/Kg/day of preterm formula or human milk. During the study, the infant received the study product or placebo as described above. When the newborn has successfully absorbed the feed, the daily feed was increased by 10-25 ml/Kg/day to complete full feed of 140-160 ml/Kg/day or study day 28, or until discharge day (if released before day 28), Table 7 below summarizes the feeding protocol details:

TABLE 7
Feeding Protocol Details

| | Age | | | | |
|---|---|---|---|---|---|
| | | | | | Day 5 & |
| | Day 1 | Day 2 | Day 3 | Day 4 | on |
| Total fluids | 80 | 100 | 120 | 140 | 150 |
| (enteral. + | ml/Kg/day | ml/Kg/day | ml/Kg/day | ml/Kg/day | ml/Kg/day |
| intra- | | | | | |
| venous + | | | | | |
| arterial line | | | | | |
| fluids) | | | | | |
| IV | 2.0 | 3.0 | 3.5 | 3.5-4.0 | 3.5-4.0 |
| Aminoacids | g/Kg/day | g/Kg/day | g/Kg/day | g/Kg/day | g/Kg/day |
| IV Fat - | *0.5 | 1.0 | 2.0 | 2.5 | 3.0 |
| Clinoleic/ | g/Kg/day | g/Kg/day | g/Kg/day | g/Kg/day | g/Kg/day |
| Lipofundin/ | | | | | |
| Intralipid | | | | | |

*Not all the infants get fat from day 1 in the NICU

### Statistical Methods

All measured variables and derived parameters were tabulated by descriptive statistics. Categorical variables were presented in summary tables including sample size, absolute and relative frequencies by study group and overall. Continuous variables were presented in summary tables including sample size, arithmetic mean, standard deviation, median, minimum and maximum by study group and overall.

The following statistical tests were used in the analysis of the data presented in this study:

The Paired T-Test was applied for testing the statistical significance of the changes from baseline for quantitative variables within each study group.

The two-sample T-test and Non-parametric Wilcoxon Rank Sum test was applied for testing differences between the study groups for quantitative parameters.

Chi-square test was applied for testing the statistical significance of the differences in frequency of categorical variables between the study groups.

Area Under the Curve (AUC) was calculated for absolute as well as relative weight changes from day 1.

All tests applied were two-tailed, and p value of 5% or less was considered statistically significant. The data was analyzed using the SAS® version 9.1 (SAS Institute, Cary N.C.).

### Results

### Demographic Data

The distribution of subjects' demographic and baseline weight is provided in tables 8-9 below. Of the 34 subjects, 46.9% were males (53.3% and 41.2% in control and treatment groups, respectively); mean age at the first day of the study was around 5 days in both study groups (range 1-7 days); mean weight at birth was 1470.7 and 1464.3 gr in treatment group and placebo group respectively. No statistically significant differences between groups were observed (P-value=0.9570. The average number of days of treatment 24.7 and 25.5 in control and treatment group respectively. No statistically significant difference was observed between the study groups (P-value=0.5887).

TABLE 8
Demographic Data

| | Control | | Treatment | | All | |
|---|---|---|---|---|---|---|
| | No. of | | No. | | No. of | |
| Demographic data | subjects | % | of subjects | % | subjects | % |
| Gender | | | | | | |
| Female | 7 | 46.7 | 10 | 58.8 | 17 | 53.1 |
| Male | 8 | 53.3 | 7 | 41.2 | 15 | 46.9 |
| Single/Twin/Triple | | | | | | |
| Singleton | 6 | 40.0 | 6 | 35.3 | 12 | 37.5 |
| Triplets | 1 | 6.7 | 2 | 11.8 | 3 | 9.4 |
| Twin | 8 | 53.3 | 9 | 52.9 | 17 | 53.1 |

TABLE 9
Birth Weight

| Birth Weight (gr) | Control | InsuMeal ™ |
|---|---|---|
| No. of subjects | 15 | 17 |
| Mean | 1464.3 | 1470.7 |
| Std | 370.6 | 299.7 |
| Min | 920.0 | 850.0 |
| Median | 1620.0 | 1474.0 |
| Max | 1960.0 | 1980.0 |

### Insulin Effect of Body Weight

A. Insulin Effect on Body Weight at Infant Age of 28 Days

Mean results for the change in weight relative to the weight at the first day (day 1) are presented in Table 10. The results demonstrate a greater increase in weight from day 1 to day 28 in the InsuMeal™ receiving group compared to the placebo receiving group.

TABLE 10
Chang in weight by visits, all infants

| Change in Weight from Weight at Day 1 (gr) | | Control | InsuMeal ™ |
|---|---|---|---|
| Day 20 | No. of subjects | 10 | 13 |
| | Mean | 487.0 | 563.9 |
| | Std | 223.9 | 155.2 |
| | Median | 415.0 | 576.0 |
| | Min | 230.0 | 260.0 |
| | Max | 950.0 | 875.0 |
| Day 25 | No. of subjects | 8 | 11 |
| | Mean | 663.8 | 735.6 |
| | Std | 271.1 | 175.4 |
| | Median | 605.0 | 760.0 |
| | Min | 410.0 | 440.0 |
| | Max | 1150 | 1010 |
| Day 28 | No. of subjects | 5 | 6 |
| | Mean | 887.0 | 892.5 |
| | Std | 269.2 | 153.1 |
| | Median | 870.0 | 865.5 |
| | Min | 580.0 | 720.0 |
| | Max | 1305 | 1155 |

Similar results were obtained for sub-group analyses of infants with birth weight below 1,300 gr (Table 11). Here, the a greater increase in weight from day 1 to day 28 in the InsuMeal™ receiving group compared to the placebo receiving group was even more pronounced.

TABLE 11
Change in weight by visits, infant birth weight < 1300 gr

| Change in weight from weight at day 1 (gr) | | Control | InsuMeal ™ |
|---|---|---|---|
| Day 20 | No. of subjects | 5 | 4 |
| | Mean | 366.0 | 531.3 |
| | Std | 119.3 | 66.88 |
| | Median | 370.0 | 532.5 |
| | Min | 230.0 | 450.0 |
| | Max | 520.0 | 610.0 |
| Day 25 | No. of subjects | 5 | 4 |
| | Mean | 525.0 | 716.3 |
| | Std | 156.0 | 102.9 |
| | Median | 430.0 | 722.5 |
| | Min | 410.0 | 590.0 |
| | Max | 755.0 | 830.0 |
| Day 28 | No. of subjects | 3 | 3 |
| | Mean | 733.3 | 778.0 |
| | Std | 145.7 | 50.86 |
| | Median | 750.0 | 799.0 |
| | Min | 580.0 | 720.0 |
| | Max | 870.0 | 815.0 |

B. Insulin Effect on Body Weight at Infant Age of Three Months

The difference between the control and assay groups with respect to the weight gained during the first 3 months was based on the difference Z3-Z0, where Z0 is the Z value at birth and Z3 is the Z value at 3 months. The mean and 95% confidence limit (CL) were evaluated for each age and for the gained weight. These data are presented in Table 12. The mean gain in the InsuMeal™ group was about twice as that of the Placebo group (0.76 vs. 0.32 SDs). The difference between the two treatment groups is significant at P=0.077.

TABLE 12
Mean and 95% of Z value (of weight) at birth
and at three months age in each group.

| | | At birth | At 3 months | Gain |
|---|---|---|---|---|
| Group | N | (Z0) | (Z3) | (Z3 − Z0) |
| Placebo | 9 | −3.077 | −2.755 | 0.323 |
| | | (−3.509; −2.645) | (−2.950; −1.923) | (−0.164; 0.809) |
| InsuMeal ™ | 10 | −3.194 | −2.436 | 0.758 |
| | | (−3.538; −2.850) | (−2.950; −1.923) | (0.301; 1.215) |
| Difference | | −0.117 | 0.318 | 0.436 |
| | | (−0.622; 0.387) | (−0.488; 1.125) | (−0.181; 1.053) |

These results support the finding presented in Example 1 hereinabove, showing that feeding low birth weight infants, including preterm infants with insulin-enriched formula accelerates the infant weight gain during the crucial first months of life, leading the normal development thereafter.

### Insulin Effect on Gastrointestinal Maturation

Additional goal of the study was to examine the effect of orally administered insulin on gastrointestinal maturation of preterm, low weight birth infants. The measure for gastrointestinal maturation was the number of days required to achieve complete enteral feeding, defined as orally consuming infant formula at a volume of at least 150 ml/Kg/day. Mean number of days to achieve complete enteral feeding was 6.4 in the insulin receiving group as compared to 7.9 in the placebo group (Table 13). The results demonstrate a trend of reduced time required to reach full enteral feed in the treatment group as compared to the control group, but with no statistical significance (P-value=0.2085).

TABLE 13
Number of days to achieve complete enteral feeding
(>=150 ml/Kg/day), all Infants

| Number of days | Control | InsuMeal ™ |
|---|---|---|
| No. of subjects | 15 | 17 |
| Mean | 7.9 | 6.4 |
| Std | 3.5 | 3.2 |
| Min | 4.0 | 1.0 |
| Median | 7.0 | 6.0 |
| Max | 17.0 | 11.0 |

Sub-group analyses of infants with birth weight <1300 gr (Table 14) and of infant presented by gender (data not shown) showed similar results as for the analysis performed for all infants.

TABLE 14
Number of days to achieve complete enteral feeding (>=150
ml/Kg/day), infants having birth weight of < 1300 gr

| Number of days | Control | InsuMeal ™ |
|---|---|---|
| No. of subjects | 6 | 5 |
| Mean | 8.8 | 8.0 |
| Std | 4.2 | 3.9 |
| Min | 6.0 | 2.0 |
| Max | 17.0 | 11.0 |

Infant maturation and growth was further assessed by the number of hospitalization days required until the preterm infant could be discharged. A trend for lower number of days to discharge was observed in the insulin-receiving group: 28.8 days as compared to 33.6 days in the group receiving the placebo treatment (Table 15). The difference did not reach statistical significance (P-value=0.2192).

TABLE 15
Number of days to discharge, all infants

| Number of days to discharge | Control | InsuMeal ™ |
|---|---|---|
| No. of subjects | 15 | 17 |
| Mean | 33.6 | 28.8 |
| Std | 12.6 | 8.8 |
| Min | 14.0 | 16.0 |
| Median | 32.0 | 28.0 |
| Max | 54.0 | 50.0 |

Sub-group analysis for infants with birth weight <1300 gr demonstrated, however, a statistically significant difference in the number of days to discharge: 35.8 days for the insulin receiving group and 47.0 for the placebo receiving (Table 16; P value=0.0447).

TABLE 16
Number of days to discharge, infant birth weight < 1300 gr

| Number of days to discharge | Control | InsuMeal ™ |
|---|---|---|
| No. of subjects | 6 | 5 |
| Mean | 47.0 | 35.8 |
| Std | 5.1 | 10.4 |
| Min | 39.0 | 28.0 |
| Median | 48.0 | 29.0 |
| Max | 54.0 | 50.0 |

The results presented above clearly demonstrate the beneficial effect of insulin given at a concentration range mimicking that of human breast colostrum and milk on the growth rate of low birth weight infant. In particular, the growth enhancement during the first one to six months of the low birth weight infant lead to an early closure of the developmental gap, reaching the growth rate of normal-weight born infant early on, thus avoiding the long term complications typically associated with low birth weight. No deleterious effects resulting from the insulin administration were observed.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-26. (canceled)

27. A method for enhancing the growth rate of a low birth weight human infant, comprising orally administering insulin to the infant when newborn, thereby enhancing the growth rate of said infant over the expected growth rate.

28. The method of claim 27, wherein the low birth weight infant is selected from the group consisting of a preterm human infant and small for gestational age (SGA) human infant.

29. The method of claim 27, wherein enhancing the growth rate comprises a measure above the expected for the low birth weight infant of at least one of the infant weight, height, head circumference and any combination thereof.

30. The method of claim 29, wherein the measure is taken at an infant age selected from the group consisting of at least 1 month, at least 3 months and six months.

31. The method of claim 27, wherein the insulin is encapsulated in an encapsulating matrix.

32. The method of claim 27, wherein the insulin is mixed with an infant formula to form an insulin-enriched formula.

33. The method of claim 32, wherein the insulin-enriched formula comprises insulin at a concentration range of from 50 μIU/ml to 600 μIU/ml.

34. The method of claim 31, wherein the encapsulated insulin is mixed with an infant formula to form an insulin-enriched formula.

35. The method of claim 34, wherein the insulin-enriched formula comprises insulin at a concentration range of from 50 μIU/ml to 600 μIU/ml.

36. The method of claim 27, wherein enhancing the growth rate comprises a measure above the expected for the low weight birth infant for gastrointestinal maturation.

37. The method of claim 36, wherein the measure of the infant gastrointestinal maturation is set by the number of days required to achieve complete enteral feed.

38. The method of claim 37, wherein the number of days required to achieve complete enteral feed is reduced compared to the expected number of days.

39. The method of claim 27, wherein enhancing the growth rate result in reducing the period of hospitalization of the infant compared to the expected hospitalization period.

40. The method of claim 27, wherein the insulin is biologically active.

41. The method of claim 27, wherein the insulin is mammalian insulin selected from the group consisting of human insulin and bovine insulin.

42. The method of claim 41, wherein the insulin is human insulin.

43. The method of claim 42, wherein the human insulin is selected from the group consisting of recombinant human insulin and semi-synthetic human insulin.

44. The method of claim 27, wherein the insulin is administered via a route selected from normal feeding and a nasogastric tube.

45. The method of claim 31, wherein the encapsulated insulin is administered via a route selected from normal feeding and a nasogastric tube.

46. The method of claim 27, wherein the insulin is administered during at least the first month after gestation up to during six months after gestation.
